(19) United States
(12) Patent Application Publication
(10) Pub. No.: US 2022/0187232 A1
(43) Pub. Date: Jun. 16, 2022

(54) GAS SENSOR AND METHOD OF SELECTIVELY DETECTING ACETYLENE AND ETHYLENE

(71) Applicant: Eberhard Karls Universität Tübingen, Tübingen (DE)

(72) Inventors: Abdulaziz Alharbi, Tübingen (DE); Nicolae Barsan, Tübingen (DE); Udo Weimar, Tübingen (DE)

(21) Appl. No.: 17/603,984

(22) PCT Filed: Apr. 15, 2020
(86) PCT No.: PCT/DE2020/000078

§ 371 Date: Oct. 15, 2021

(30) Foreign Application Priority Data
Apr. 16, 2019 (DE) 10 2019 002 782.5

(51) Int. Cl. G01N 27/12; G01N 33/00

(52) U.S. Cl. CPC G01N 27/125; G01N 33/2841; G01N 33/0047; G01N 27/123

(57) ABSTRACT

A gas sensor that selectively detects and/or measures acetylene and/or ethylene includes a substrate; at least one electrode pair; at least one gas-sensitive layer consisting of at least one metal oxide from the group ReFeO₃ and in contact with the at least one electrode pair; a heating element; and at least one control device, wherein the heating element is adapted to be heated alternately to at least two different temperatures of 150° C.-250° C., 200° C.-300° C. and 250° C.350° C., respectively, by the control device.

### TECHNICAL FIELD

This disclosure relates to a gas sensor and also a method of selectively or jointly detecting and/or measuring acetylene and/or ethylene.

### BACKGROUND

Gas sensors are used in many different fields of application, for example, in medicine or industry to detect the presence of a particular gas and/or to determine its concentration.

Owing to their cost efficiency and robustness, gas sensors based on metal oxides are widely used. Many gas sensors which use both single metal oxides and also mixed oxides are known. On contact of such materials with particular gases, the conductivity of the metal oxide changes and is measured and evaluated by downstream electronics to identify the corresponding gases and concentration. The conductivity of the gas-sensitive metal oxide is temperature dependent, for which reason a heating element is additionally provided in many gas sensors.

In PCT/EP2006/010892, the use of various metal oxides including metal oxides having a perovskite structure from the group consisting of LaFeO₃ and SmFeO₃ is proposed for the gas-sensitive layer. The gas sensor of PCT/EP2006/010892 is said to be able to be used to measure hydrogen, carbon monoxide, alcohol, hydrocarbons, ammonia, amines, oxygen, nitrogen monoxide and nitrogen dioxide.

The disadvantages of the gas sensors based on metal oxide, thus also the gas sensor described in PCT/EP2006/010892, include in particular the cross-sensitivity and low selectivity, especially in measurements of structurally similar gases. For that reason, they are used predominantly in measuring instruments for which a low accuracy suffices.

Frequently, a number of gases are present in the measurement that can lead to false alarms or a falsified measurement because the sensor is not able to distinguish various gases. For example, a regular measurement of gases dissolved in transformer oil (C₂H₂, C₂H₄, CH₄, C₂H₆, CO, CO₂ and H₂) is used to evaluate the state of power transformers. Acetylene (C₂H₂) plays an important role since an increased concentration can be a sign of damaged insulation.

Great efforts have been made to increase the selectivity of gas sensors to acetylene, but sensitivity to the structurally similar ethylene has always been observed at the same time in Q. Zhang, Q. Zhou, Z. Lu, Z. Wei, L. Xu, Y. Gui, Recent Advances of SnO₂-Based Sensors for Detecting Fault Characteristic Gases Extracted from Power Transformer Oil, Front. Chem. 29 (2018) 1-7, doi: 10.3389/fchem.2018.00364 and L. Jin, W. Chen, H. Zhang, G. Xiao, C. Yu, Characterization of Reduced Graphene Oxide (rGO)-Loaded SnO₂ Nanocomposite and Applications in C₂H₂ Gas Detection, Appl. Sci. 7 (2017) 19, doi: 10.3390/app7010019. The selective recognition and monitoring of acetylene and ethylene thus remain a great challenge in many industrial applications.

It could therefore be helpful to provide a gas sensor and also a method of selectively or jointly detecting and/or measuring acetylene and/or ethylene.

### SUMMARY

We provide a gas sensor that selectively detects and/or measures acetylene and/or ethylene including a substrate, at least one electrode pair, at least one gas-sensitive layer consisting of at least one metal oxide from the group ReFeO₃ and in contact with the at least one electrode pair, a heating element, and at least one control device, wherein the heating element is adapted to be heated alternately to at least two different temperatures of 150° C.-250° C., 200° C.-300° C. and 250° C.-350° C., respectively, by the control device.

We also provide a gas sensor that selectively detects and/or measures acetylene and/or ethylene including a substrate, at least one electrode pair, at least one gas-sensitive layer consisting of at least one metal oxide from the group ReFeO₃ and is in contact with the at least one electrode pair, two or more heating elements, and at least one control device, wherein each heating element is adapted to be heated to at least one particular different temperature of 150° C.-250° C., 200° C.-300° C. and 250° C.-350° C., respectively.

We further provide a method of selectively detecting and/or measuring acetylene and/or ethylene at a gas-sensitive layer consisting of at least one metal oxide from the group ReFeO₃, including measuring temperatures alternately at at least two different temperatures of 150° C.-250° C., 200° C.-300° C. and 250° C.-350° C., respectively.

### BRIEF DESCRIPTION OF THE DRAWINGS

FIGS. 1(A)-(C) show schematic structures of the LFO sensor elements from above, below and in cross section, with a sensor layer (1), interdigital electrodes (2), a substrate (3) and a heating element (4).

FIGS. 2(A)-(B) show schematic structures of the gas sensor including a gas-sensitive layer having a compact (A) or porous (B) structure in comparison.

FIG. 3 shows sensor signals (R_{gas}/Rₐᵢᵣ) for different gases (1: C₂H₂, 2: C₂H₄, 3: CO₂, 4: CO, ₅: H_{2, 6}: CH₄), measured at the LFO sensor at a temperature of 200° C., shown on a logarithmic scale.

FIG. 4 shows sensor signals (R_{gas}/Rₐᵢᵣ) for different gases (1: C₂H₂, 2: C₂H₄, 3: CO₂, 4: CO, 5: H_{2,} 6: CH₄), measured at the LFO sensor at a temperature of 250° C., shown on a logarithmic scale.

FIG. 5 shows sensor signals (R_{gas}/Rₐᵢᵣ) for different concentrations of acetylene and ethylene (25, 50, 100, 300, 500, 1000, 1500, 3000 and 5000 ppm), measured at the LFO sensor at a temperature of 200° C., shown on a logarithmic scale. Curve 1 shows the sensor signals in the presence of acetylene, curve 2 shows the sensor signals in the presence of ethylene (each 25, 50, 100, 300, 500, 1000, 1500, 3000 and 5000 ppm).

FIG. 6 shows sensor signals (R_{gas}/Rₐᵢᵣ) for different concentrations of acetylene and ethylene (25, 50, 100, 300, 500, 1000, 1500, 3000 and 5000 ppm), measured at the LFO sensor at a temperature of 250° C., shown on a logarithmic scale. Curve 1 shows the sensor signals in the presence of acetylene, curve 2 shows the sensor signals in the presence of ethylene (each 25, 50, 100, 300, 500, 1000, 1500, 3000 and 5000 ppm).

FIG. 7 shows sensor signals (R_{gas}/Rₐᵢᵣ) for different concentrations of acetylene and ethylene (25, 50, 100, 300, 500, 1000, 1500, 3000 and 5000 ppm), measured at the LFO sensor at a temperature of 300° C., shown on a logarithmic scale. Curve 1 shows the sensor signals in the presence of acetylene, curve 2 shows the sensor signals in the presence of ethylene (each 25, 50, 100, 300, 500, 1000, 1500, 3000 and 5000 ppm).

### DETAILED DESCRIPTION

Our gas sensor comprises a metal oxide as gas-sensitive layer and either a heating element that can be heated alternately to at least two different temperatures by a control device or comprises a plurality of heating elements that can each be heated to at least one particular temperature by at least one control device. The temperatures to which the heating elements can be heated are each different.

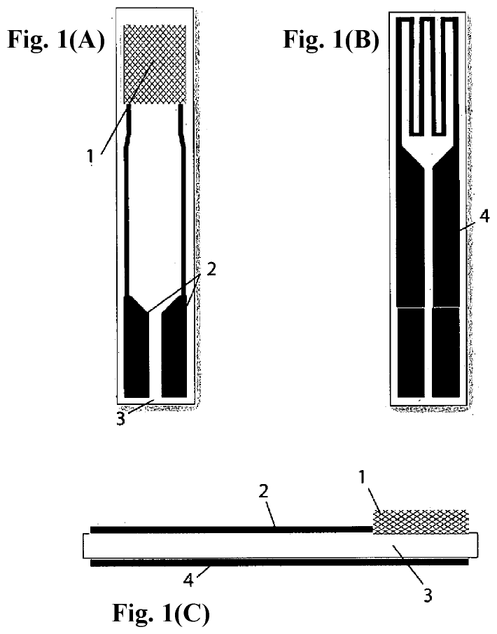

A schematic structure of an illustrative gas sensor is shown in FIGS. 1(A)-(C).

The bandwidth of the temperatures to which the heating elements of the sensor can be heated is 150° C. to 350° C.

In a simplest working example, only one heating element that can be heated to two particular temperatures of 150° C.-250° C. and 200° C.-300° C., respectively, is provided. A non-limiting example is a gas sensor having a heating element that can be heated alternately to either 200° C. or 250° C.

In another working example, a heating element that can be heated to three particular temperatures of 150° C.-250° C., 200° C.-300° C. and 250° C.-350° C., respectively, is provided. A nonlimiting example is a gas sensor having a heating element that can be heated alternately to 200° C., 250° C. or 300° C.

Use of heating elements that can be heated to four or more particular temperatures selected and defined for the respective purpose is likewise encompassed by this disclosure.

In a further example, the gas sensor has two or more heating elements, with each heating element being able to be heated to a particular temperature.

In one working example having two heating elements, the one heating element can be heated to a temperature of 150° C.-250° C. and the other heating element can be heated to a temperature of 200° C.-300° C. A nonlimiting example is a gas sensor having two heating elements and in which the first heating element can be heated to a temperature of 200° C. and the second heating element can be heated to a temperature of 250° C.

In another working example, the gas sensor has three heating elements that can be heated to three particular temperatures of 150° C.-250° C., 200° C.-300° C. and 250° C.-350° C., respectively. A nonlimiting example is a gas sensor having three heating elements that can be heated to 200° C., 250° C. and 300° C., respectively.

Use of four or more heating elements that can be heated to four or more particular temperatures selected and defined for the respective purpose is likewise encompassed by this disclosure.

The metal oxide in the gas-sensitive layer is preferably selected from the group ReFeO₃, where Re is a metal of the rare earth elements.

The metal oxide is preferably selected from the group consisting of LaFeO₃, SmFeO₃, EuFeO₃ and GdFeO₃. It can be a pure or doped metal oxide. Particular preference is given to using LaFeO₃ (LFO) for the gas-sensitive layer.

The ratio of Re:Fe in the gas-sensitive layer is preferably approximately 1:1, e.g., with a maximum deviation of 10%.

The material for the gas-sensitive layer which is particularly suitable for the gas sensor is preferably produced by the sol-gel method and is calcined at a temperature of 500° C. to 900° C., e.g., at 600° C. To produce the gas-sensitive layer, the material that has been prepared in this way is mixed with a solvent and the resulting paste is deposited on electrodes by screen printing.

As an alternative, the gas-sensitive layer can also be produced by a flame spray pyrolysis process.

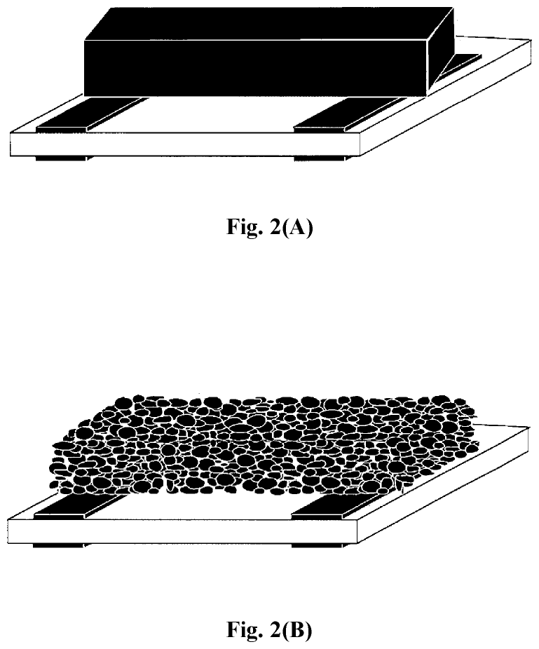

Preferably, the gas-sensitive layer is applied in the form of nanostructures in contact with one another on the substrate as shown in FIGS. 2(A)-(B) and described in Q. Zhang, Q. Zhou, Z. Lu, Z. Wei, L. Xu, Y. Gui, Recent Advances of SnO₂-Based Sensors for Detecting Fault Characteristic Gases Extracted from Power Transformer Oil, Front. Chem. 29 (2018) 1 -7, doi: 10.3389/fchem.2018.00364.

The nanostructures that form the gas-sensitive layer have a high homogeneity in terms of shape and size and can be produced, for example, by the two abovementioned methods. They preferably have dimensions of less than a few microns, in particular 10 nm to 100 nm. The nanostructures preferably have a uniform size with a maximum deviation of 10%, in particular not more than 5%. By way of example and not constituting a restriction, the nanoparticles can have the shape of spheres or nanorods.

The substrate can consist of ceramic, MEMS or another known material. As electrodes, it is possible to use, for example, interdigital electrodes based on the noble metals.

We also provide a measurement method in which the gas measurement or gas detection is carried out alternately at at least two different temperatures. The temperature range in which measurements are carried out is, in particular, 150° C. to 350° C. Measurement data from the measurements at at least two different temperatures are used for the evaluation of the measurement results and determination of the gas concentration. The order of the gas measurement or gas detection at various temperatures can be different.

The method can be carried out by the above-described gas sensor.

In one working example, the gas measurement or gas detection is carried out alternately at only two defined temperatures of 150° C.-250° C. and 200° C.-300° C., respectively, e.g., alternately at 200° C. and 250° C.

In another working example, the gas measurement or the gas detection is carried out at three different temperatures of 150° C.-250° C., 200° C.-300° C. and 250° C.-350° C., respectively. To name a specific, nonlimiting example, the gas measurement or gas detection can be carried out alternately at 200° C., 250° C. and 300° C.

The gas measurement or gas detection can also be carried out at four or more different temperatures.

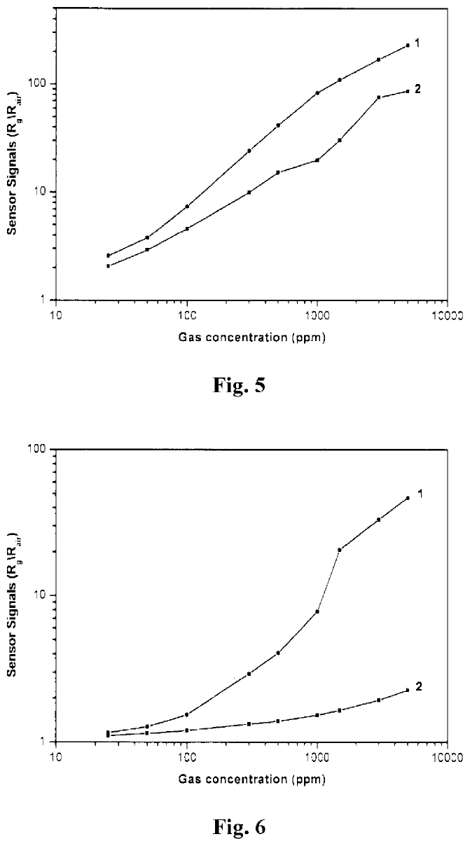

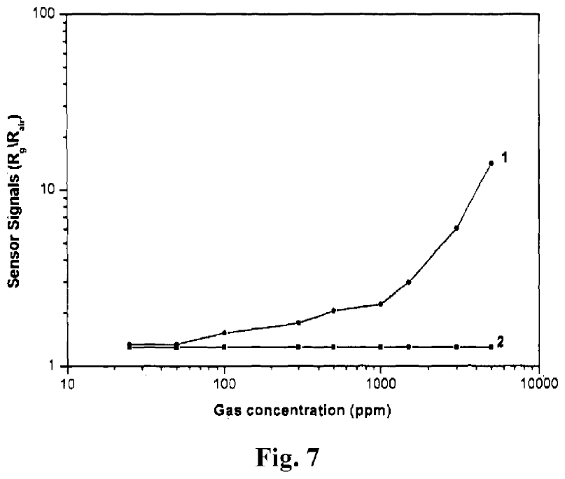

The measurement at more than two temperatures can be advantageous when, for example, one or more characteristic curves for the gases to be measured or detected are to be constructed so that precise conclusions as to the gases present and their concentrations can be drawn therefrom as shown in FIGS. 5, 6 and 7.

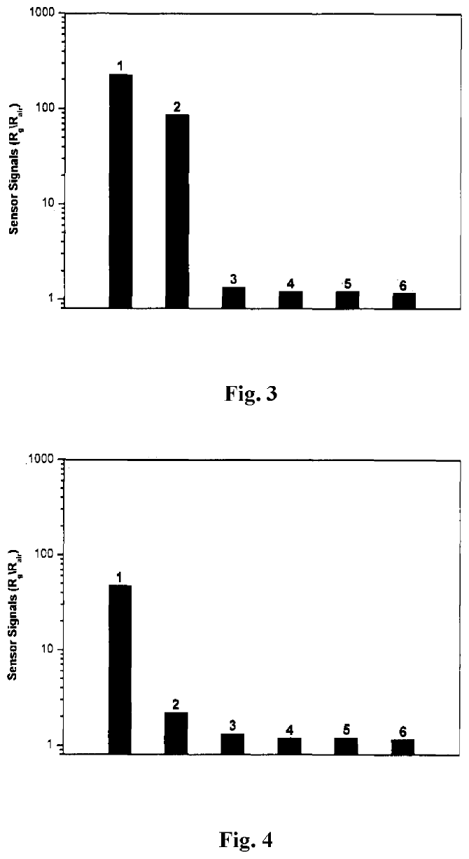

In a measurement using the sensor at a measurement temperature of about 200° C., the sensor signal for acetylene behaves similar to the sensor signal for ethylene as shown in FIG. 3 so that identification of the measured gas or making a distinction between the two gases is virtually impossible on the basis of only this measurement.

In a second measurement at a temperature of about 250° C., the sensor reacts very selectively to acetylene, virtually without cross-reaction to ethylene as shown in FIG. 4. Thus, the sensor signal for acetylene at this temperature is more than 20 times higher than for ethylene (at a gas concentration of 5000 ppm).

The alternating measurement at at least two different temperatures thus makes it possible to distinguish between these two related gases: when only acetylene is present, both measurements, at 200° C. and at 250° C., each give a relatively strong sensor signal; when only ethylene is present, the measurement at 200° C. gives a moderate sensor signal and the measurement at 250° C. gives a very weak sensor signal. Precise conclusions as to the concentrations of gases present can be drawn, for example, with the aid of characteristic curves.

Our gas sensors and methods thus for the first time allow highly selective detection, distinguishing and quantification of ethylene and acetylene individually and in gas mixtures. The sensors have no or only very little sensitivity (cross-sensitivity) to other gases (e.g., CO₂, CO, H₂ and CH₄).

Owing to the high selectivity, the gas sensors and methods can advantageously be used for, in particular, detecting and/or measuring acetylene and/or ethylene, e.g., in transformer oil, to determine ethylene to establish the degree of ripeness of fruit or vegetables or measure and regulate the ethylene concentration in the ripening chambers.

Further advantages, features and possible uses are described below with the aid of the working examples set forth below with reference to the figures. Working examples

### Synthesis of Materials

LFO perovskite materials (LaFeO₃) were synthesized using various chemical methods including solid-state reaction and sol-gel process. Stoichiometric and nonstoichiometric structures were also prepared. In some samples, partial replacement of iron by highly charged cations such as tungsten was carried out. All materials formed were calcined at various temperatures of 500° C. to 900° C. Various techniques and manipulations of a plurality of parameters were used to obtain sensor layers based on perovskite having particular properties, for example, surface homogeneity, nanostructure and high porosity.

During preparation, perovskites are highly reactive toward CO₂ and atmospheric moisture. For this reason, hydroxylation and formation of carbonates, mainly formed on the surface, occur in the ambient air during production of such materials. Although treatments at high temperatures are necessary, these lead to a reduction in the reactive surface area which is undesirable for the gas measurement. It is therefore important to establish an equilibrium between, on the one hand, the hydroxylation and carbonates resulting therefrom and, on the other hand, provision of a relatively large homogeneous surface area.

To achieve this objective, two synthetic approaches were used:

In a solid-state reaction, fixed proportions of the oxides La₂O₃ and Fe₂O₃ (and other oxides as dopants) were used to produce pure (or doped) LaFeO₃. NaHCO₃ was added in a ratio of (5:1) to the total amount of metal oxide and the mixture was subsequently calcined at various temperatures of 500° C. to 900° C. in an aluminum oxide crucible for 10 hours. The resulting powders were cooled to room temperature and washed with distilled water to remove all traces of sodium compounds. Finally, the powders were dried at 70° C. for 12 hours. The single-phase perovskite crystal structure was obtained only in the powders that had been produced at temperatures of 700° C. and above. They had the orthorhombic crystal system according to the Inorganic Crystal Structure Database (ICSD). The reference numbers of the conforming samples are (28255).

In a sol-gel process, the starting substances La(NO₃)₃·6H₂O, Fe(NO₃)₃·6H₂O (and dopants, e.g., WCl₆) and also citric acid were used to produce LFO-based materials. The citric acid was added in a ratio of 1:1 to the total amount of metal nitrates, the mixture was dissolved in deionized water and subsequently mixed using magnetic stirrers for one hour. The solution was then neutralized to a pH of about 6-7 by addition of ammonium hydroxide and stirred further overnight. The resulting gel was dried at 90° C. for 4 hours and subsequently calcined at various temperatures from 500 to 900° C. in an aluminum oxide crucible for 2 hours. The powders obtained were examined by X-ray diffraction to confirm that the products have a perovskite crystal structure at all calcination temperatures of 500° C. to 900° C.

The powders obtained from all the materials prepared in this way were used to produce gas-sensitive layers by mixing them with a solvent to obtain a printable paste that was then deposited over interdigital platinum electrodes by screen printing. The sensors obtained in this way were dried overnight at 70° C. Finally, they were calcined using a heating sequence in three steps (400° C., 500° C. and 400° C.) each for 10 minutes. The schematic structure of the LFO sensor elements is depicted in FIGS. 1(A)-(C). The sensor layer (1) and the interdigital electrodes (2) are arranged on the upper side of the aluminum oxide substrate (3). The heating element (4) applied to the underside of the substrate serves to heat the sensitive layer to the desired operating temperature. In addition, the cross section of the sensor is shown to demonstrate the thickness of various components.

The structure of the sensor material can, as a function of its morphology, play a key role in the mechanism of the gas sensor system. FIGS. 2(A)-(B) show two forms of the sensor layer, namely the compact layer (A) and the porous layer (B). In the compact layer, the interaction with gases takes place only at the geometric surface because gases cannot penetrate into the sensitive layer. In a porous layer, the gases can come into contact with the entire surface area of the layer because the active surface area is much greater than in the compact layer. This also affects the conduction process during exposure to gas, as is comprehensively explained in N. Barsan, U. Weimar, Conduction model of metal oxide gas sensors, J. Electroceramics 7 (2001 143 -167, doi: 10.1023/A:1014405811371.

To produce particularly porous surfaces, the flame spray pyrolysis process can also be used, as described in J. A. Kemmler, S. Pokhrel, L. Mädler. U. Weimar and N. Barsan, Flame Spray Pyrolysis for Sensing at the Nanoscale, Nanotechnology 24 (2013) 1 -14, doi: 10.1088/0957-4484/24/44/442001.

### Gas Measurement

The gas sensor materials having the LFO perovskite structure synthesized by the above-described methods were examined in respect of their gas sensor properties. Surprisingly, the best gas sensor performance in respect of the sensitivity and the selectivity was displayed by the material that had been produced by the sol-gel method and calcined at 600° C. (LFO 600). Some of the materials displayed a similar selectivity behavior, but with lower sensitivity, and the others had cross-sensitivity with other gases.

Results of the measurements using two sensors having an LFO perovskite structure are shown in comparison in Table 1, with the material of the one sensor having been produced by the sol-gel process and the material of the other sensor in a solid-state reaction. Selective measurement was observed only in the sensor material produced by the sol-gel process, at an operating temperature of 250° C. The more selective material had far smaller particles (about 50 nm) than the less selective material (1-2 μm). In addition, the more selective material produced by the sol-gel process had a higher surface homogeneity with a significantly smaller amount of carbonates than the material synthesized in a solid-state reaction, as our spectroscopic studies showed.

TABLE 1
The signals measured by two LFO sensors produced by different
methods after exposure to 5000 ppm of acetylene (C₂H₂) and ethylene
(C₂H₄) at measurement temperatures of 200° C. and 250° C.

| | Sensor signals (R_{gas}/Rₐᵢᵣ) | | | |
|---|---|---|---|---|
| | 200° C. | | 250° C. | |
| | C₂H₂ | C₂H₄ | C₂H₂ | C₂H₄ |
| LaFeO₃ | (5000 ppm) | (5000 ppm) | (5000 ppm) | (5000 ppm) |
| Produced by | 229 | 86 | 47 | 2.2 |
| sol-gel | | | | |
| process | | | | |
| Produced using | 8.30 | 13.13 | 19.78 | 14.65 |
| solid-state | | | | |
| reaction | | | | |

A significant improvement in the selectivity of the LFO sensor for unsaturated hydrocarbons, in particular for acetylene and ethylene, was thus achieved by selection of the sensor material and by adaptation of the measurement temperature. Thus, our LFO sensor displayed a similar reaction to acetylene and ethylene with higher sensor signals for acetylene than for ethylene at a measurement temperature of 200° C. However, the sensor displayed, as quite different property, very little reaction to CO_{2,} CO, H₂ and CH₄ as shown in FIG. 3.

At a relatively high operating temperature of 250° C., this sensor became very selective for acetylene, with virtually no reaction to ethylene. At this temperature, the sensor signal for acetylene is more than 20 times higher than for ethylene, at a concentration of 5000 ppm as shown in FIG. 4. At temperatures above 250° C., the LFO sensor displayed an even greater selectivity for acetylene, but with lower sensor signals.

FIGS. 5, 6 and 7 show sensor signals measured at the LFO sensor at different concentrations (25, 50, 100, 300, 500, 1000, 1500, 3000 and 5000 ppm) of acetylene and ethylene at 200° C., 250° C. and 300° C. respectively. Curve 1 shows the sensor signals in the presence of acetylene. Curve 2 shows the sensor signals in the presence of ethylene under otherwise identical conditions.

To examine the cross-sensitivity between acetylene and ethylene, the sensor was exposed to the two gases individually and at the same time at different concentrations as shown in Table 2. The measurements were carried out at five different concentrations (500, 1000, 1500, 3000 and 5000 ppm) at different operating temperatures (200° C., 250° C. and 300° C.).

TABLE 2
Procedure for the measurements

| First cycle (only | Second cycle (only | Third cycle (both |
|---|---|---|
| ethylene) ppm | acetylene) ppm | gases) ppm |
| 500 | 500 | 500 C₂H₂ + 500 C₂H₄ |
| 1000 | 1000 | 1000 C₂H₂ + 1000 C₂H₄ |
| 1500 | 1500 | 1500 C₂H₂ + 1500 C₂H₄ |
| 3000 | 3000 | 3000 C₂H₂ + 3000 C₂H₄ |
| 5000 | 5000 | 5000 C₂H₂ + 5000 C₂H₄ |

The cross-sensitivity of our LFO sensor to acetylene relative to ethylene is calculated using equation (1):

$$The cross \text{-} sensitivity at a concentration = ( \frac{S for both gases - S for only acetylene}{S for only ethylene} ) * 100 , \qquad ( 1 )$$

where S is the sensor signal.

The average cross-sensitivity of our LFO sensor to acetylene relative to ethylene for different concentrations at each measurement temperature is shown in Table 3.

TABLE 3
Average cross-sensitivity of the gas sensor to acetylene
relative to ethylene at various measurement temperatures

| Operating temperature | Average cross-sensitivity |
|---|---|
| 200° C. | 16.95% |
| 250° C. | 4.63% |
| 300° C. | 5.06% |

1-19. (canceled)

20. A gas sensor that selectively detects and/or measures acetylene and/or ethylene comprising:
a substrate;
at least one electrode pair;
at least one gas-sensitive layer consisting of at least one metal oxide from the group ReFeO₃ and in contact with the at least one electrode pair;
a heating element; and
at least one control device, wherein the heating element is adapted to be heated alternately to at least two different temperatures of 150° C.-250° C., 200° C.-300° C. and 250° C.-350° C., respectively, by the control device.

21. A gas sensor that selectively detects and/or measures acetylene and/or ethylene comprising:
a substrate;
at least one electrode pair;
at least one gas-sensitive layer consisting of at least one metal oxide from the group ReFeO₃ and in contact with at least one electrode pair;
two or more heating elements; and
at least one control device, wherein each heating element is adapted to be heated to at least one particular different temperature of 150° C.-250° C., 200° C.-300° C. and 250° C.-350° C., respectively.

22. The gas sensor as claimed in claim 20, wherein the metal oxide is selected from the group consisting of LaFeO₃, SmFeO₃, EuFeO₃ or GdFeO₃.

23. The gas sensor as claimed in claim 20, wherein a ratio of Re:Fe in the gas-sensitive layer is approximately 1:1, with a maximum deviation of 10%.

24. The gas sensor as claimed in claim 20, wherein the metal oxide for the gas-sensitive layer is produced by a sol-gel method and calcined at a temperature of 500° C. to 900° C., or is produced by an FSP method.

25. The gas sensor as claimed in claim 20, wherein the metal oxide has been applied in the form of nanostructures that touch one another on the substrate.

26. The gas sensor as claimed in claim 25, wherein the nanostructures have the shape of spheres or rods.

27. The gas sensor as claimed in claim 25, wherein the nanostructures have a size of less than 10 μm.

28. The gas sensor as claimed in claim 21, wherein the heating element is adapted to be heated to two particular temperatures of 150° C.-250° C. and 200° C.-300° C., respectively.

29. The gas sensor as claimed in claim 21, wherein the heating element is adapted to be heated to three particular temperatures of 150° C.-250° C., 200° C.-300° C. and 250° C.-350° C., respectively.

30. The gas sensor as claimed in claim 28, comprising two heating elements that have a first heating element adapted to be heated to a temperature of 150° C.-250° C. and a second heating element adapted to be heated to a temperature of 200° C.-300° C.

31. The gas sensor as claimed in claim 29, comprising three heating elements that have a first heating element adapted to be heated to a temperature of 150° C.-250° C., a second heating element adapted to be heated to a temperature of 200° C.-300° C. and a third heating element adapted to be heated to a temperature of 250° C.-350° C.

32. The gas sensor as claimed in claim 20, wherein the substrate comprises ceramic and/or MEMS.

33. The gas sensor as claimed in claim 20, that detects and/or measures gases in transformer oil or determines a degree of ripeness of fruit or vegetables or to determines a gas concentration in a ripening chamber.

34. A method of selectively detecting and/or measuring acetylene and/or ethylene at a gas-sensitive layer consisting of at least one metal oxide from the group ReFeO₃, comprising measuring temperatures alternately at at least two different temperatures of 150° C.-250° C., 200° C.-300° C. and 250° C.-350° C., respectively.

35. The method as claimed in claim 34, wherein measurement data from the measurements at at least two different temperatures evaluate the measurement results and determine the gas concentration.

36. The method as claimed in claim 35, wherein the gas measurement or gas detection is carried out alternately at two defined temperatures of 150° C.-250° C. and 200° C.-300° C., respectively.

37. The method as claimed in claim 34, wherein the gas measurement or gas detection is carried out alternately at three different temperatures of 150° C.-250° C., 200° C.-300° C. and 250° C.-350° C., respectively.

38. The method as claimed in claim 34 to determine a degree of ripeness of fruit or vegetables or determines the gas concentration in a ripening chamber.
